(19) United States
(12) Patent Application Publication
(10) Pub. No.: US 2007/0004954 A1
(43) Pub. Date: Jan. 4, 2007

(54) Method for producing liquefied petroleum gas

(76) Inventors: Sachio Asaoka, Kitakyushu-shi (JP); Xiahong Li, Kitakyushu-shi (JP); Kaoru Fujimoto, Kitakyushu-shi (JP); Kenji Asami, Kitakyushu (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/545,896

(22) PCT Filed: Feb. 18, 2004
(86) PCT No.: PCT/JP04/01804

§ 371 Date: Jun. 13, 2006

(30) Foreign Application Priority Data
Feb. 18, 2003 (JP) 2003-039591
Oct. 16, 2003 (JP) 2003-356488

(51) Int. Cl. C07C 1/00

(52) U.S. Cl. 585/733

(57) ABSTRACT

A liquefied petroleum gas containing propane or butane as a main component is produced by passing a raw material gas comprising hydrogen and at least one selected from the group consisting of methanol and dimethyl ether through a catalyst layer comprising, in the direction of flowing of the raw material gas, a former catalyst layer comprising a catalyst for synthesizing an olefin-containing gas which is used when producing an olefin-containing gas from at least one selected from the group consisting of methanol and dimethyl ether, and a latter catalyst layer comprising a catalyst for hydrogenating an olefin-containing gas which is used when an olefin is hydrogenated to produce a paraffin.

### TECHNICAL FIELD

This invention relates to a process for producing a liquefied petroleum gas containing propane or butane as a main component from at least one selected from the group consisting of methanol and dimethyl ether.

### BACKGROUND ART

Liquefied petroleum gas (LPG) is a liquefied petroleum-based or natural-gas-based hydrocarbon which is gaseous at an ambient temperature under an atmospheric pressure by compression while optionally cooling, and the main component of it is propane or butane. LPG is advantageously transportable because it can be stored or transported in a liquid form. Thus, in contrast with a natural gas that requires a pipeline for supply, it has a characteristic that it can be filled in a container to be supplied to any place. For that reason, LPG comprising propane as a main component, i.e., propane gas has been widely used as a fuel for household and business use. At present, propane gas is supplied to about 25 million households (more than 50% of the total households) in Japan. In addition to household and business use, LPG is used as a fuel for a portable product such as a portable gas burner and a disposable lighter (mainly, butane gas), an industrial fuel and an automobile fuel.

Conventionally, LPG has been produced by 1) collection from a wet natural gas, 2) collection from a stabilization (vapor-pressure regulating) process of crude petroleum, 3) separation and extraction of a product in, for example, a petroleum refining process, or the like.

LPG, in particular propane gas used as a household/business fuel, can be expected to be in great demand in the future. Thus, it may be very useful to establish an industrially practicable and new process for producing LPG.

As a process for producing LPG, “Selective Synthesis of LPG from Synthesis Gas”, Kaoru Fujimoto et al., Bull. Chem. Soc. Jpn., 58, p. 3059-3060 (1985) discloses that, using a hybrid catalyst consisting of a methanol synthesis catalyst such as a 4 wt % Pd/SiO₂, a Cu—Zn—Al mixed oxide {Cu:Zn:Al=40:23:37 (atomic ratio)} or a Cu-based low-pressure methanol synthesis catalyst (Trade name: BASF S3-85) and a high-silica Y-type zeolite with SiO₂/Al₂O₃=7.6, C2 to C4 paraffins can be produced in a selectivity of 69 to 85% via methanol and dimethyl ether from a synthesis gas. However, in the process, the selectivity of propane (C3) and butane (C4) is about 63 to 74%, and the product of the process may not be suitable for LPG products.

Furthermore, butane is the main component of the product obtained by the process described in the above-mentioned “Selective Synthesis of LPG from Synthesis Gas”, Bull. Chem. Soc. Jpn., 58, p. 3059-3060 (1985). As described above, propane gas is the LPG used as a fuel for household and business use. Propane gas has the advantage that it can be continuously combusted with a stably higher power at low temperature in comparison with butane gas. Propane gas is superior to butane gas as a liquefiable fuel gas, which is widely used as a household/business fuel and as an industrial fuel and an automobile fuel, because propane gas has a sufficient, higher vapor pressure in winter or in a cold region and generates higher calories during combustion.

### DISCLOSURE OF THE INVENTION

An objective of this invention is to provide a process for producing a liquefied petroleum gas containing propane or butane as a main component from at least one selected from the group consisting of methanol and dimethyl ether.

Another objective of this invention is to provide a process for more economically producing a liquefied petroleum gas containing propane or butane as a main component from at least one selected from the group consisting of methanol and dimethyl ether.

The present invention provides a process for producing a liquefied petroleum gas, comprising a step of:

producing a liquefied petroleum gas containing propane or butane as a main component from hydrogen and at least one selected from the group consisting of methanol and dimethyl ether by a catalytic reaction.

Furthermore, the present invention provides a process for producing a liquefied petroleum gas, comprising a step of:

producing a liquefied petroleum gas containing propane or butane as a main component by passing a raw material gas comprising hydrogen and at least one selected from the group consisting of methanol and dimethyl ether through a catalyst layer (the 1^{st} process for producing LPG).

Moreover, the present invention provides the above process for producing a liquefied petroleum gas, wherein the catalyst layer comprising, in the direction of flowing of the raw material gas,

a former catalyst layer comprising a catalyst for synthesizing an olefin-containing gas which is used when producing an olefin-containing gas from at least one selected from the group consisting of methanol and dimethyl ether, and

a latter catalyst layer comprising a catalyst for hydrogenating an olefin-containing gas which is used when an olefin is hydrogenated to produce a paraffin.

Moreover, the present invention provides the above process for producing a liquefied petroleum gas, wherein the catalyst layer comprising, in the direction of flowing of the raw material gas,

a former catalyst layer comprising a catalyst for synthesizing an olefin-containing gas which is used when producing an olefin-containing gas from at least one selected from the group consisting of methanol and dimethyl ether,

a middle catalyst layer comprising a zeolite catalyst component and a catalyst component for hydrogenating an olefin-containing gas which is used when an olefin is hydrogenated to produce a paraffin, and

a latter catalyst layer comprising a catalyst for hydrogenating an olefin-containing gas which is used when an olefin is hydrogenated to produce a paraffin.

Furthermore, the present invention provides a process for producing a liquefied petroleum gas, comprising:

(1) a step of producing an olefin-containing gas wherein a raw material gas comprising hydrogen and at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component, water and hydrogen; and

(2) a step of hydrogenating an olefin-containing gas wherein the reaction gas obtained in the step of producing an olefin-containing gas is passed through a catalyst layer comprising a catalyst for hydrogenating an olefin-containing gas which is used when an olefin is hydrogenated to produce a paraffin, to produce a liquefied petroleum gas containing propane or butane as a main component (the 2-1^{st} process for producing LPG).

Moreover, the present invention provides a process for producing a liquefied petroleum gas, comprising:

(1) a step of producing an olefin-containing gas wherein a raw material gas comprising hydrogen and at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component, water and hydrogen; and

(2) a step of isomerizing and hydrogenating an olefin-containing gas wherein the reaction gas obtained in the step of producing an olefin-containing gas is passed through a catalyst layer comprising a zeolite catalyst component and a catalyst component for hydrogenating an olefin-containing gas which is used when an olefin is hydrogenated to produce a paraffin, to produce a liquefied petroleum gas containing propane or butane as a main component (the 2-2^{nd} process for producing LPG).

Moreover, the present invention provides a process for producing a liquefied petroleum gas, comprising:

(1) a step of producing an olefin-containing gas wherein a raw material gas comprising hydrogen and at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component, water and hydrogen;

(2) a step of isomerizing and hydrogenating an olefin-containing gas wherein the reaction gas obtained in the step of producing an olefin-containing gas is passed through a catalyst layer comprising a zeolite catalyst component and a catalyst component for hydrogenating an olefin-containing gas which is used when an olefin is hydrogenated to produce a paraffin, to produce a reaction gas containing propylene or butene as a main component and propane or butane and hydrogen; and

(3) a step of hydrogenating an olefin-containing gas wherein the reaction gas obtained in the step of isomerizing and hydrogenating an olefin-containing gas is passed through a catalyst layer comprising a catalyst for hydrogenating an olefin-containing gas which is used when an olefin is hydrogenated to produce a paraffin, to produce a liquefied petroleum gas containing propane or butane as a main component (the 2-3^{rd} process for producing LPG).

Furthermore, the present invention provides a process for producing a liquefied petroleum gas, comprising:

(1) a step of producing an olefin-containing gas wherein a raw material gas comprising at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component and water; and

(2) a step of hydrogenating an olefin-containing gas wherein the reaction gas obtained in the step of producing an olefin-containing gas and a hydrogen-containing gas are passed through a catalyst layer comprising a catalyst for hydrogenating an olefin-containing gas which is used when an olefin is hydrogenated to produce a paraffin, to produce a liquefied petroleum gas containing propane or butane as a main component (the 3-1^{st} process for producing LPG).

Moreover, the present invention provides a process for producing a liquefied petroleum gas, comprising:

(1) a step of producing an olefin-containing gas wherein a raw material gas comprising at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component and water; and

(2) a step of isomerizing and hydrogenating an olefin-containing gas wherein the reaction gas obtained in the step of producing an olefin-containing gas and a hydrogen-containing gas are passed through a catalyst layer comprising a zeolite catalyst component and a catalyst component for hydrogenating an olefin which is used when an olefin is hydrogenated to produce a paraffin, to produce a liquefied petroleum gas containing propane or butane as a main component (the 3-2^{nd} process for producing LPG).

Moreover, the present invention provides a process for producing a liquefied petroleum gas, comprising:

(1) a step of producing an olefin wherein a raw material gas comprising at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component and water;

(2) a step of isomerizing and hydrogenating an olefin-containing gas wherein the reaction gas obtained in the step of producing an olefin-containing gas and a hydrogen-containing gas are passed through a catalyst layer comprising a zeolite catalyst component and a catalyst component for hydrogenating an olefin which is used when an olefin is hydrogenated to produce a paraffin, to produce a reaction gas containing propylene or butene as a main component and propane or butane and hydrogen; and

(3) a step of hydrogenating an olefin-containing gas wherein the reaction gas obtained in the step of isomerizing and hydrogenating an olefin-containing gas is passed through a catalyst layer comprising a catalyst for hydrogenating an olefin-containing gas which is used when an olefin is hydrogenated to produce a paraffin, to produce a liquefied petroleum gas containing propane or butane as a main component (the 3-3^{rd} process for producing LPG).

Furthermore, the present invention provides a process for producing a liquefied petroleum gas, comprising:

(1) a step of producing an olefin-containing gas wherein an olefin-containing gas comprising propylene or butene as a main hydrocarbon component as well as ethylene is produced from at least one selected from the group consisting of methanol and dimethyl ether and the ethylene-containing fraction which is separated from the olefin-containing gas in the step of separation and recycled as a starting material for the step of producing an olefin-containing gas in the step of recycling, in the presence of a catalyst for synthesizing an olefin-containing gas which is used when producing an olefin-containing gas from at least one selected from the group consisting of methanol and dimethyl ether;

(2) a step of separation wherein an ethylene-containing fraction is separated from the olefin-containing gas obtained in the step of producing an olefin-containing gas, to obtain a propylene-containing fraction;

(3) a step of recycling wherein a part or all of the ethylene-containing fraction separated in the step of separation is recycled as a starting material for the step of producing an olefin-containing gas; and

(4) a step of hydrogenating an olefin-containing gas wherein a liquefied petroleum gas containing propane or butane as a main component is produced from hydrogen and the propylene-containing fraction obtained in the step of separation, in the presence of a catalyst for hydrogenating an olefin-containing gas which is used when an olefin is hydrogenated to produce a paraffin (the 4^{th} process for producing LPG).

According to this invention, a liquefied petroleum gas containing propane or butane as a main component can be produced from at least one selected from the group consisting of methanol and dimethyl ether.

According to this invention, a liquefied petroleum gas with the content of propane of 50 to 100% on the basis of carbon, for example, can be produced. And, according to this invention, a liquefied petroleum gas with the total content of propane and butane of 90 to 100% on the basis of carbon, for example, can be produced.

In the fourth process for producing LPG according to this invention, after an olefin-containing gas comprising propylene or butene as a main component is produced from at least one selected from the group consisting of methanol and dimethyl ether, ethylene is separated from the obtained olefins, and recycled as a starting material for the step of producing an olefin-containing gas.

When producing an olefin-containing gas from at least one selected from the group consisting of methanol and dimethyl ether, carbene (H₂C:), which is generated by dehydration of methanol, is polymerized to form an olefin, so that there are generally provided two or more types of olefins having a certain composition distribution, not a single type of olefin. When producing olefins, the main component of which is propylene having three carbon atoms, the resulting olefin-containing gas (reaction gas) comprises, in addition to propylene, ethylene having two carbon atoms, butene having four carbon atoms, and the like.

On the other hand, when ethylene is added to at least one selected from the group consisting of methanol and dimethyl ether as a starting material of the reaction, an olefin-containing gas comprising propylene or butene as a main component is generally obtained, without significantly changing the composition distribution in the resulting olefin-containing gas.

Thus, the final yield of propylene and/or butene may increase by separating ethylene from an olefin-containing gas produced and recycling the ethylene as a starting material for the step of producing an olefin-containing gas. Therefore, propylene and/or butene, and furthermore propane and/or butane can be produced in a higher yield from at least one selected from the group consisting of methanol and dimethyl ether.

### BRIEF DESCRIPTION OF THE DRAWINGS

FIG. 1 is a process flow diagram showing a main configuration in an example of an LPG producing apparatus suitable for conducting the 1st process for LPG production according to this invention.

FIG. 2 is a process flow diagram showing a main configuration in an example of an LPG producing apparatus suitable for conducting the 2-1st process for LPG production according to this invention.

FIG. 3 is a process flow diagram showing a main configuration in an example of an LPG producing apparatus suitable for conducting the 3-1st process for LPG production according to this invention.

FIG. 4 is a process flow diagram showing a main configuration in an example of an LPG producing apparatus suitable for conducting the 4th process for LPG production according to this invention.

### DESCRIPTION OF THE MAIN SYMBOLS

11: a reactor
11a: a catalyst layer comprising a catalyst for synthesizing an olefin-containing gas
11b: a catalyst layer comprising a catalyst for hydrogenating an olefin-containing gas
13, 15: lines.
21: a first reactor
21a: a catalyst layer comprising a catalyst for synthesizing an olefin-containing gas
22: a second reactor
22a: a catalyst layer comprising a catalyst for hydrogenating an olefin-containing gas
23, 24, 25: lines.
31: a first reactor
31a: a catalyst layer comprising a catalyst for synthesizing an olefin-containing gas
32: a second reactor
32a: a catalyst layer comprising a catalyst for hydrogenating an olefin-containing gas
33, 34, 35, 36: lines.
41: a first reactor
41a: a catalyst for synthesizing an olefin-containing gas
42: a separator
43: a second reactor
43a: a catalyst for hydrogenating an olefin-containing gas
411, 412, 413, 415, 416, 417: lines
414: a recycle line.

### BEST MODE FOR CARRYING OUT THE INVENTION

In the 1st process for producing LPG according to this invention, a raw material gas comprising hydrogen and at least one selected from the group consisting of methanol and dimethyl ether is passed through a catalyst layer to produce a liquefied petroleum gas containing propane or butane as a main component.

The catalyst layer may, for example, comprise, in the direction of flowing of the raw material gas, a former catalyst layer comprising a catalyst for synthesizing an olefin-containing gas which is used when producing an olefin-containing gas from at least one selected from the group consisting of methanol and dimethyl ether, and a latter catalyst layer comprising a catalyst for hydrogenating an olefin-containing gas which is used when an olefin is hydrogenated to produce a paraffin.

Alternatively, the catalyst layer may comprise, in the direction of flowing of the raw material gas, a former catalyst layer comprising a catalyst for synthesizing an olefin-containing gas which is used when producing an olefin-containing gas from at least one selected from the group consisting of methanol and dimethyl ether, a middle catalyst layer comprising a zeolite catalyst component and a catalyst component for hydrogenating an olefin which is used when an olefin is hydrogenated to produce a paraffin, and a latter catalyst layer comprising a catalyst for hydrogenating an olefin-containing gas which is used when an olefin is hydrogenated to produce a paraffin.

In the 2-1st process for producing LPG according to this invention, first a raw material gas comprising hydrogen and at least one selected from the group consisting of methanol and dimethyl ether is passed through a catalyst layer comprising a catalyst for synthesizing an olefin-containing gas to obtain a reaction gas comprising an olefin mixture containing propylene or butene as a main component, water and hydrogen (the step of producing an olefin-containing gas), and then the reaction gas obtained in the previous step is passed through a catalyst layer comprising a catalyst for hydrogenating an olefin-containing gas to produce a liquefied petroleum gas containing propane or butane as a main component (the step of hydrogenating an olefin-containing gas).

The 2-1st process for producing LPG may comprise, instead of the step of hydrogenating an olefin-containing gas, the step of isomerizing and hydrogenating an olefin-containing gas wherein the reaction gas obtained in the step of producing an olefin-containing gas is passed through a catalyst layer comprising a zeolite catalyst component and a catalyst component for hydrogenating an olefin to produce a liquefied petroleum gas containing propane or butane as a main component (the 2-2nd process for producing LPG).

The 2-1st process for producing LPG may comprise, instead of the step of hydrogenating an olefin-containing gas, the step of isomerizing and hydrogenating an olefin-containing gas wherein the reaction gas obtained in the step of producing an olefin-containing gas is passed through a catalyst layer comprising a zeolite catalyst component and a catalyst component for hydrogenating an olefin to produce a reaction gas containing propylene or butene as a main component and propane or butane and hydrogen, and the subsequent step of hydrogenating an olefin-containing gas wherein the reaction gas obtained in the step of isomerizing and hydrogenating an olefin-containing gas is passed through a catalyst layer comprising a catalyst for hydrogenating an olefin-containing gas to produce a liquefied petroleum gas containing propane or butane as a main component (the 2-3rd process for producing LPG).

In the 3-1st process for producing LPG according to this invention, first a raw material gas comprising at least one selected from the group consisting of methanol and dimethyl ether is passed through a catalyst layer comprising a catalyst for synthesizing an olefin-containing gas to obtain a reaction gas comprising an olefin mixture containing propylene or butene as a main component (the step of producing an olefin-containing gas), and then the reaction gas obtained in the previous step and a hydrogen-containing gas are passed through a catalyst layer comprising a catalyst for hydrogenating an olefin-containing gas to produce a liquefied petroleum gas containing propane or butane as a main component (the step of hydrogenating an olefin-containing gas).

The 3-1st process for producing LPG may comprise, instead of the step of hydrogenating an olefin-containing gas, the step of isomerizing and hydrogenating an olefin-containing gas wherein the reaction gas obtained in the step of producing an olefin-containing gas and a hydrogen-containing gas are passed through a catalyst layer comprising a zeolite catalyst component and a catalyst component for hydrogenating an olefin to produce a liquefied petroleum gas containing propane or butane as a main component (the 3-2nd process for producing LPG).

The 3-1st process for producing LPG may comprise, instead of the step of hydrogenating an olefin-containing gas, the step of isomerizing and hydrogenating an olefin-containing gas wherein the reaction gas obtained in the step of producing an olefin-containing gas and a hydrogen-containing gas are passed through a catalyst layer comprising a zeolite catalyst component and a catalyst component for hydrogenating an olefin to produce a reaction gas containing propylene or butene as a main component and propane or butane and hydrogen, and the subsequent step of hydrogenating an olefin-containing gas wherein the reaction gas obtained in the step of isomerizing and hydrogenating an olefin-containing gas is passed through a catalyst layer comprising a catalyst for hydrogenating an olefin-containing gas to produce a liquefied petroleum gas containing propane or butane as a main component (the 3-3rd process for producing LPG).

Herein, a “zeolite catalyst component” means a zeolite which can act as a catalyst in a reaction of methanol into a hydrocarbon and/or a reaction of dimethyl ether into a hydrocarbon.

In the process for producing LPG according to this invention, a reaction raw material may be methanol or dimethyl ether alone, or may be a mixture of methanol and dimethyl ether. When using a mixture of methanol and dimethyl ether as a reaction raw material, a ratio of methanol to dimethyl ether is not particularly limited. And, a crude methanol comprising water, or the like can be used as a reaction raw material.

Dimethyl ether can be produced from methanol and used as a reaction raw material.

In the above processes for producing LPG, the catalyst layer comprising a catalyst for synthesizing an olefin-containing gas may comprise two or more types of catalysts for synthesizing an olefin-containing gas. If the reaction can be controlled so that the main component of the resulting olefins may be propylene or butene, the catalyst layer comprising a catalyst for synthesizing an olefin-containing gas may comprise one or more types of catalysts for hydrogenating an olefin-containing gas. There may be two or more catalyst layers comprising a catalyst for synthesizing an olefin-containing gas. In the catalyst layer comprising a catalyst for synthesizing an olefin-containing gas, the composition may change in regard to the direction of flowing of the raw material gas.

The catalyst for synthesizing an olefin-containing gas may be one having catalytic function for hydrogenating an olefin. Examples of such a catalyst include a catalyst having both a zeolite catalyst component and a catalyst component for hydrogenating an olefin; specifically, a catalyst in which a catalyst for synthesizing an olefin-containing gas consisting of a zeolite catalyst component, which is described later, is modified (supported, ion-exchanged, skeletal substituted or mixing of these metal components supported on another carrier) with a metal such as Fe, Ni, Pd and Pt as a catalyst component for hydrogenating an olefin.

In the above processes for producing LPG, the catalyst layer comprising a catalyst for hydrogenating an olefin-containing gas may comprise two or more types of catalysts for hydrogenating an olefin-containing gas. And, the catalyst layer comprising a catalyst for hydrogenating an olefin-containing gas may comprise one or more types of catalysts for synthesizing an olefin-containing gas. There may be two or more catalyst layers comprising a catalyst for hydrogenating an olefin-containing gas. In the catalyst layer comprising a catalyst for hydrogenating an olefin-containing gas, the composition may change in regard to the direction of flowing of the raw material gas.

The catalyst for hydrogenating an olefin-containing gas may be one having catalytic function for synthesizing an olefin-containing gas. Such a catalyst is the same as the catalyst for synthesizing an olefin-containing gas having catalytic function for hydrogenating an olefin.

In the above processes for producing LPG, the catalyst layer comprising a zeolite catalyst component and a catalyst component for hydrogenating an olefin may comprise two or more types of zeolite catalyst components, and may comprise two or more types of catalyst components for hydrogenating an olefin. The catalyst layer comprising a zeolite catalyst component and a catalyst component for hydrogenating an olefin may be a mixture of a zeolite catalyst component and a catalyst component for hydrogenating an olefin, or may comprise a catalyst having both a zeolite catalyst component and a catalyst component for hydrogenating an olefin. There may be two or more catalyst layers comprising a zeolite catalyst component and a catalyst component for hydrogenating an olefin. In the catalyst layer comprising a zeolite catalyst component and a catalyst component for hydrogenating an olefin, the composition may change in regard to the direction of flowing of the raw material gas.

A catalyst layer may comprise an additive component other than a catalyst component for synthesizing an olefin-containing gas (a zeolite catalyst component) and a catalyst component for hydrogenating an olefin. For example, a catalyst may be diluted with quartz sand and the like to form a catalyst layer.

In the above processes for producing LPG, LPG containing propane or butane as a main component may be produced from at least one selected from the group consisting of methanol and dimethyl ether, following the formula (I) shown below.

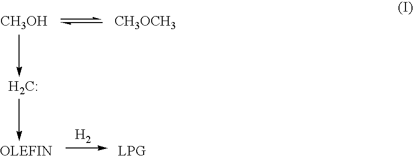

In the present invention, probably, methanol is dehydrated to generate a carbene (H₂C:) by a concerted catalysis of an acidic site and a basic site, which are at a spatial field inside a pore in a zeolite as a catalyst for synthesizing an olefin-containing gas. And then, the carbene is polymerized to form ethylene as a dimer; propylene as a trimer or a reaction product with ethylene; and butylene as a tetramer, a reaction product with propylene or a product of dimerization of ethylene. A carbene is probably generated by decomposition of ethylene and the like.

In the olefin formation process, there may occur some reactions such as formation of dimethyl ether by dehydration-dimerization of methanol; formation of methanol by hydration of dimethyl ether; formation of a higher olefin by polymerization of a lower olefin; decomposition of a higher olefin; cyclization of an olefin; formation of an aromatic hydrocarbon, a conjugated hydrocarbon compound and a saturated hydrocarbon by isomerization; and tar or coke formation from a conjugated hydrocarbon compound having a cyclopentadienyl structure or the like.

In this invention, it is important to inhibit, among the above reactions, the reactions other than the reactions for forming an olefin and paraffin which have a carbon number corresponding to a desired LPG or their precursor, i.e., a reaction to form carbene, a reaction to form a lower olefin such as ethylene, propylene and butene by polymerization of carbene, a reaction between carbene and ethylene or propylene, a dimerization reaction of ethylene, and a decomposition reaction of a higher olefin. It is also important to control the reactions so that the main component of the formed olefin mixture may be propylene or butene.

For that purpose, it is important to use a zeolite having an appropriate acid strength, an appropriate amount of acid (acid concentration) and an appropriate pore size as a catalyst for synthesizing an olefin-containing gas and/or a zeolite catalyst component.

Examples of a catalyst for synthesizing an olefin-containing gas and a zeolite catalyst component include ZSM-34; ZSM-5; preferably high-silica ZSM-5 with a Si/Al ratio (atomic ratio) of 100 or less; a silicoaluminophosphate (SAPO) such as SAPO-34; ECR-1; mazmorite; and a synthetic paulingite-type zeolite such as ECR-18. Examples of a catalyst for synthesizing an olefin-containing gas and a zeolite catalyst component also include the above zeolites containing a metal such as Ni, Co, Fe, Pt, Pd, Cu and Ag or an element such as Mg, P and a lanthanide, and the above zeolites ion-exchanged with any of these metals, these elements, Ti, Nb and the like. An acid strength and an amount of acid of a zeolite can be adjusted by introducing a metal or a compound; ion-exchanging with a metal or a compound; or depositing a coke. Furthermore, an acid strength and an amount of acid of a zeolite can be adjusted not only averagely but also specifically, for example, in the outside of a zeolite pore, in the vicinity of a pore opening and in the inside of a pore. In addition to adjustment of an acid strength and an amount of acid, a pore size can be finely adjusted at once or separately. Coke may be deposited, in addition to introduction of a metal or a compound or ion-exchanging with a metal or a compound. Among them, a preferable catalyst for synthesizing an olefin-containing gas or a preferable zeolite catalyst component are high-silica ZSM-5 and SAPO-34, more preferably ZSM-5 with a Si/Al ratio (atomic ratio) of 100 or less, more preferably 20 to 70 (both inclusive), or a metallosilicate with MFI structure wherein half or less of Al in the skeleton is substituted with Fe.

For producing an olefin mixture containing propylene or butene as a main component, it is also important to control the reaction conditions, particularly a contact time of a raw material gas with a catalyst for synthesizing an olefin-containing gas. Since an olefin-forming reaction such as polymerization of carbene and polymerization of olefin is a successive reaction, there is a tendency that as a contact time of a raw material gas with a catalyst for synthesizing an olefin-containing gas is longer, the resulting olefins have more carbon atoms.

A contact time of a raw material gas with a catalyst for synthesizing an olefin-containing gas to provide an olefin-containing gas containing propylene or butene as a main component varies depending on the type of the catalyst used, the other reaction conditions and the like. In this invention, a reaction for synthesizing an olefin-containing gas may be conducted beforehand to determine a contact time of a raw material gas with a catalyst for synthesizing an olefin-containing gas.

As described above, a catalyst layer comprising a catalyst for hydrogenating an olefin-containing gas may comprise a catalyst for synthesizing an olefin-containing gas in this invention. However, in this case, it is necessary to determine a contact time of a raw material gas with a catalyst for synthesizing an olefin-containing gas so that a reaction to form an olefin having more carbon atoms, i.e., a reaction to consume an olefin having a carbon number corresponding to a desired LPG, can be inhibited, considering the fact that the reactions such as polymerization of carbene and polymerization of olefin proceed in a catalyst layer comprising a catalyst for hydrogenating an olefin-containing gas.

And, when using a catalyst layer comprising a zeolite catalyst component and a catalyst component for hydrogenating an olefin-containing gas, it is necessary to determine a contact time of a raw material gas with a catalyst for synthesizing an olefin-containing gas and/or a zeolite catalyst component so that a reaction to form an olefin having more carbon atoms, i.e., a reaction to consume an olefin having a carbon number corresponding to a desired LPG, can be inhibited, considering the fact that the reactions such as polymerization of carbene and polymerization of olefin proceed in the catalyst layer.

On the other hand, examples of a catalyst for hydrogenating an olefin-containing gas and a catalyst component for hydrogenating an olefin include known hydrogenation catalysts and known catalyst components for hydrogenating an olefin; specifically, metals such as Fe, Co, Ni, Ru, Rh, Pd, Os, Ir, Pt, Cu and Re, and alloys thereof, oxides of metals such as Cu, Co, Ni, Cr, Zn, Re, Mo and W; sulfides of metals such as Co, Re, Mo and W. Any of these catalysts supported on a carrier such as carbon, silica, alumina, silica-alumina and a zeolite, or mixed therewith can be used. Among them, preferable catalysts for hydrogenating an olefin-containing gas include a nickel catalyst, a palladium catalyst and a platinum catalyst. Among them, preferable catalyst components for hydrogenating an olefin include Fe, Ni, Pd and Pt.

In a catalyst layer comprising a zeolite catalyst component and a catalyst component for hydrogenating an olefin, a ratio (by weight) of the catalyst component for hydrogenating an olefin to the zeolite catalyst component may be appropriately determined, but is preferably 0.5 to 1.5 in general.

In the first process for producing LPG, the catalyst layer may consist of one or more catalyst layers comprising a catalyst for synthesizing an olefin-containing gas and a catalyst for hydrogenating an olefin-containing gas, or alternatively one or more catalyst layers comprising a catalyst for synthesizing an olefin having catalytic function for hydrogenating an olefin, if the reaction can be controlled so that the main component of the resulting olefins may be propylene or butene.

Next, there will be described an embodiment of a process for producing LPG according to this invention (the 1st, the 2-1st or the 3-1st process for producing LPG) with reference to the drawings.

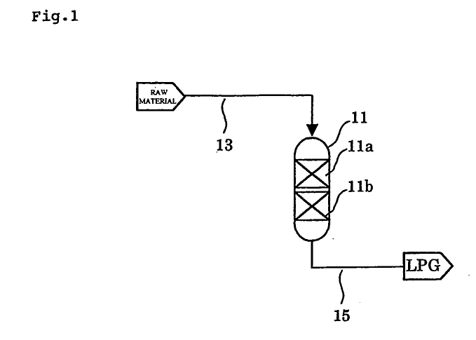

FIG. 1 shows an embodiment of an LPG production apparatus suitable for carrying out the 1st process for producing LPG according to this invention.

First, methanol and/or dimethyl ether, which are reaction raw materials, and hydrogen are fed into a reactor 11 via a line 13. In the reactor 11, there are a former catalyst layer (herein, upper catalyst layer) 11a comprising a catalyst for synthesizing an olefin-containing gas and a latter catalyst layer (herein, lower catalyst layer) 11b comprising a catalyst for hydrogenating an olefin-containing gas in the direction of flowing of the raw material gas.

Concentration(s) of methanol and/or dimethyl ether in the raw material gas may be appropriately determined, depending on the type of the catalyst used, the reaction conditions and the like. And a ratio of hydrogen to methanol and/or dimethyl ether in the raw material gas may be also appropriately determined, depending on the type of the catalyst used, the reaction conditions and the like.

Methanol and/or dimethyl ether, and hydrogen may be mixed, and then fed into the reactor or, alternatively, these may be fed into the reactor separately.

The raw material gas may comprise an inert gas such as nitrogen, helium, argon and carbon dioxide, in addition to methanol and/or dimethyl ether and hydrogen. The raw material gas may comprise water.

In the reactor 11, an olefin-containing gas comprising propylene or butene as a main component is synthesized from methanol and/or dimethyl ether in the presence of the catalyst for synthesizing an olefin-containing gas. And then, in the presence of the catalyst for hydrogenating an olefin-containing gas, the produced olefins are hydrogenated to produce paraffins containing propane or butane as a main component.

The reaction is generally conducted in a fixed bed. The reaction conditions such as a composition of a raw material gas, a reaction temperature, a reaction pressure and a contact time with a catalyst may be appropriately determined, depending on the type, the performance and the shape of the used catalyst, and the like.

The synthesized paraffins are pressurized and cooled, and LPG, which is a product, is obtained from a line 15. Hydrogen and the like may be removed from the LPG by, for example, gas-liquid separation.

The LPG production apparatus may be, as necessary, provided with a booster, a heat exchanger, a valve, an instrumentation controller and so on, which are not shown.

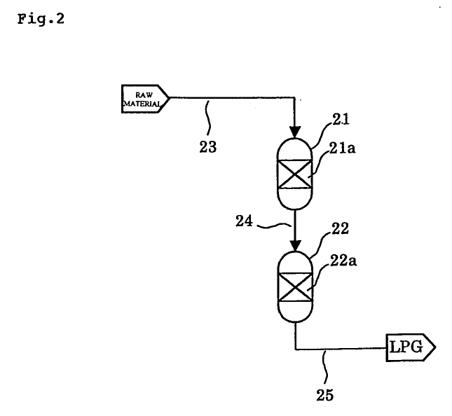

FIG. 2 shows an embodiment of an LPG production apparatus suitable for carrying out the 2-1st process for producing LPG according to this invention.

First, methanol and/or dimethyl ether, which are reaction raw materials, and hydrogen are fed into a first reactor 21 via a line 23. In the first reactor 21, there is a catalyst layer 21a comprising a catalyst for synthesizing an olefin-containing gas.

Concentration(s) of methanol and/or dimethyl ether in the raw material gas may be appropriately determined, depending on the type of the catalyst used, the reaction conditions and the like. And a ratio of hydrogen to methanol and/or dimethyl ether in the raw material gas may be also appropriately determined, depending on the type of the catalyst used, the reaction conditions and the like.

Methanol and/or dimethyl ether, and hydrogen may be mixed, and then fed into the reactor or, for removing reaction heat, these may be fed into the reactor separately; for example, methanol and/or dimethyl ether, or hydrogen can be fed into the reactor from its middle part.

The raw material gas may comprise water. The raw material gas may additionally comprise an inert gas and the like.

In the first reactor 21, an olefin-containing gas comprising propylene or butene as a main component is synthesized from methanol and/or dimethyl ether in the presence of the catalyst for synthesizing an olefin-containing gas.

The reaction can be conducted in a fixed bed, a fluidized bed or a moving bed. When there are two or more catalyst layers, it is preferable to conduct the reaction in a fixed bed. The reaction conditions such as a composition of a raw material gas, a reaction temperature, a reaction pressure and a contact time with a catalyst may be appropriately determined, depending on the type, the performance and the shape of the used catalyst, and the like.

The obtained reaction gas comprising hydrogen and an olefin mixture containing propylene or butene as a main component is fed into a second reactor 22 via a line 24. In the second reactor 22, there is a catalyst layer 22a comprising a catalyst for hydrogenating an olefin-containing gas.

A gas obtained by adding an inert gas such as nitrogen, helium and argon to the reaction gas obtained in the first reactor 21 may be fed into the second reactor 22. And, a gas obtained by adding additional hydrogen to the reaction gas obtained in the first reactor 21 may be fed into the second reactor 22.

In the second reactor 22, in the presence of the catalyst for hydrogenating an olefin-containing gas, the olefin-containing gas produced in the first reactor 21 is hydrogenated to produce paraffins containing propane or butane as a main component.

The reaction can be conducted in a fixed bed, a fluidized bed or a moving bed. When there are two or more catalyst layers, it is preferable to conduct the reaction in a fixed bed. The reaction conditions such as a reaction temperature, a reaction pressure and a contact time with a catalyst may be appropriately determined, depending on the type, the performance and the shape of the used catalyst, and the like.

The synthesized paraffins are pressurized and cooled, and LPG, which is a product, is obtained from a line 25. Hydrogen and the like may be removed from the LPG by, for example, gas-liquid separation.

The LPG production apparatus may be, as necessary, provided with a booster, a heat exchanger, a valve, an instrumentation controller and so on, which are not shown.

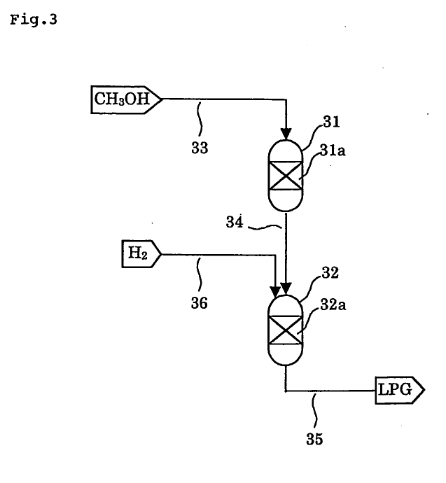

FIG. 3 shows an embodiment of an LPG production apparatus suitable for carrying out the 3-1st process for producing LPG according to this invention.

First, methanol and/or dimethyl ether, which are reaction raw materials, is fed into a first reactor 31 via a line 33. In the first reactor 31, there is a catalyst layer 31a comprising a catalyst for synthesizing an olefin-containing gas.

Concentration(s) of methanol and/or dimethyl ether in the raw material gas may be appropriately determined, depending on the type of the catalyst used, the reaction conditions and the like.

For removing reaction heat or improving the selectivity, part of methanol and/or dimethyl ether, which is a raw material gas, may be fed into the reactor separately; for example, part of methanol and/or dimethyl ether can be fed into the reactor from its middle part.

The raw material gas may comprise water. The raw material gas may additionally comprise an inert gas and the like.

In the first reactor 31, an olefin-containing gas comprising propylene or butene as a main component is synthesized from methanol and/or dimethyl ether in the presence of the catalyst for synthesizing an olefin-containing gas.

The reaction can be conducted in a fixed bed, a fluidized bed or a moving bed. When there are two or more catalyst layers, it is preferable to conduct the reaction in a fixed bed. The reaction conditions such as a composition of a raw material gas, a reaction temperature, a reaction pressure and a contact time with a catalyst may be appropriately determined, depending on the type, the performance and the shape of the used catalyst, and the like.

The obtained reaction gas comprising an olefin mixture containing propylene or butene as a main component is fed into a second reactor 32 via a line 34. And, a hydrogen-containing gas is fed into a second reactor 32 via a line 36. A hydrogen-containing gas may include hydrogen gas, and a gas obtained by adding an inert gas such as nitrogen, helium, argon and carbon dioxide to hydrogen. In the second reactor 32, there is a catalyst layer 32a comprising a catalyst for hydrogenating an olefin-containing gas.

An amount of hydrogen supplied to the reaction gas may be appropriately determined, depending on the type of the catalyst used, the reaction conditions and the like. And concentration of hydrogen in the hydrogen-containing gas may be appropriately determined.

The reaction gas and the hydrogen-containing gas may be mixed, and then fed into the second reactor 32 or, alternatively, these may be fed into the second reactor 32 separately.

In the second reactor 32, in the presence of the catalyst for hydrogenating an olefin-containing gas, the olefin-containing gas produced in the first reactor 31 is hydrogenated to produce paraffins containing propane or butane as a main component.

The reaction can be conducted in a fixed bed, a fluidized bed or a moving bed. When there are two or more catalyst layers, it is preferable to conduct the reaction in a fixed bed. The reaction conditions such as a composition of a raw material gas, a reaction temperature, a reaction pressure and a contact time with a catalyst may be appropriately determined, depending on the type, the performance and the shape of the used catalyst, and the like.

The synthesized paraffins are pressurized and cooled, and LPG, which is a product, is obtained from a line 35. Hydrogen and the like may be removed from the LPG by, for example, gas-liquid separation.

The LPG production apparatus may be, as necessary, provided with a booster, a heat exchanger, a valve, an instrumentation controller and so on, which are not shown.

Thus, LPG is produced from at least one selected from the group consisting of methanol and dimethyl ether, in this invention.

The 1st process for producing LPG is preferable in that LPG can be produced in the fewer steps. On the other hand, the 2-1st to 2-3rd and the 3-1st to 3-3rd processes for producing LPG are preferable in that the reaction can be conducted under the optimal conditions in each reaction step; separation, removal, recycling, bypassing of a certain component in a reaction gas, addition of another component, and the like can be carried out in end of each reaction step; each step can be monitored; and for each step, operation can be controlled and a catalyst can be, for example, pretreated, regenerated/activated, filled or replaced.

Next, there will be described the 4th process for producing LPG which is economically more preferable. The 4th process for producing LPG is the same as the 3-1st process for producing LPG, except that, after an olefin-containing gas comprising propylene or butene as a main component is produced from at least one selected from the group consisting of methanol and dimethyl ether, ethylene is separated from the obtained olefin-containing gas, and recycled as a starting material for the step of producing an olefin-containing gas.

In the step of producing an olefin-containing gas in the 4th process for producing LPG, in the presence of a catalyst for synthesizing an olefin-containing gas, an olefin-containing gas comprising propylene or butene as a main hydrocarbon component as well as ethylene is produced from at least one selected from the group consisting of methanol and dimethyl ether, and the ethylene-containing fraction which is separated from the olefin-containing gas in the step of separation, which is described later. The olefin-containing gas comprises water, which is a by-product, in addition to olefins and paraffins.

A reaction raw material may be methanol or dimethyl ether alone, or may be a mixture of methanol and dimethyl ether. When using a mixture of methanol and dimethyl ether as a reaction raw material, a ratio of methanol to dimethyl ether is not particularly limited. And, a crude methanol comprising water, or the like can be used as a reaction raw material.

Dimethyl ether can be produced from methanol and used as a reaction raw material.

A content of an ethylene-containing fraction in a gas fed into a reactor (hereinafter, referred to as a “raw material gas”); in other words, a content of a recycled material may be determined as appropriate and may be, for example, 10 to 50% by weight.

In the step of producing an olefin-containing gas, an olefin-containing gas comprising propylene or butene as a main component as well as ethylene may be produced from at least one selected from the group consisting of methanol and dimethyl ether, following the formula (II) shown below.

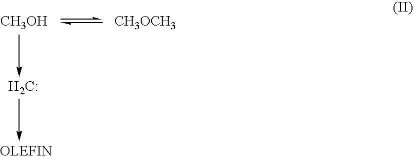

In the 4th process for producing LPG, similarly, it is important to use a zeolite having an appropriate acid strength, an appropriate amount of acid (acid concentration) and an appropriate pore size as a catalyst for synthesizing an olefin-containing gas, in order that the main component of the formed olefin mixture may be propylene or butene.

Examples of a catalyst for synthesizing an olefin-containing gas include the ones described above. In the 4th process for producing LPG, similarly, a preferable catalyst for synthesizing an olefin-containing gas is high-silica ZSM-5 and SAPO-34, more preferably ZSM-5 with a Si/Al ratio (atomic ratio) of 100 or less, more preferably 20 to 70 (both inclusive), or a metallosilicate with MFI structure wherein half or less of Al in the skeleton is substituted with Fe.

In the 4th process for producing LPG, similarly, for producing an olefin mixture containing propylene or butene as a main component, it is also important to control the reaction conditions, particularly a contact time of a raw material gas with a catalyst for synthesizing an olefin-containing gas.

A contact time of a raw material gas with a catalyst for synthesizing an olefin-containing gas to provide an olefin-containing gas containing propylene or butene as a main component varies depending on the type of the catalyst used, the other reaction conditions and the like. In this invention, a reaction for synthesizing an olefin-containing gas may be conducted beforehand to determine a contact time of a raw material gas with a catalyst for synthesizing an olefin-containing gas.

In the 4th process for producing LPG, similarly, a catalyst for synthesizing an olefin-containing gas may be used singly or in combination of two or more types. A catalyst for synthesizing an olefin-containing gas may comprise other additive components as long as its intended effect would not be impaired. For example, the above catalyst may be diluted with quartz sand and the like, and used.

There may be two or more catalyst layers comprising a catalyst for synthesizing an olefin-containing gas. In the catalyst layer comprising a catalyst for synthesizing an olefin-containing gas, the composition may change in regard to the direction of flowing of the raw material gas.

The reaction can be conducted in a fixed bed, a fluidized bed or a moving bed. When there are two or more catalyst layers, it is preferable to conduct the reaction in a fixed bed. The reaction conditions such as a composition of a raw material gas, a reaction temperature, a reaction pressure and a contact time with a catalyst may be appropriately determined, depending on the type, the performance and the shape of the used catalyst, and the like.

When using a proton-type ZSM-5 zeolite as a catalyst for synthesizing an olefin-containing gas, for example, the reaction may be carried out under the following conditions.

A gas fed into a reactor may comprise water, an inert gas and the like, in addition to at least one selected from the group consisting of methanol and dimethyl ether, and the ethylene-containing fraction which is separated from the olefin-containing gas in the step of separation described later. When using a mixture of methanol and dimethyl ether as a reaction raw material, a ratio of methanol to dimethyl ether is not particularly limited, and can be appropriately determined. A content of an ethylene-containing fraction in a gas fed into a reactor may be determined as appropriate and may be, for example, 10 to 50% by weight.

In the light of activity, an inlet temperature of a reactor is preferably 300° C or higher, more preferably 320° C. or higher. In the light of selectivity and a catalyst life, an inlet temperature of a reactor is preferably 470° C. or lower, more preferably 450° C. or lower

In the light of activity, selectivity and good operability of an apparatus, a reaction pressure is preferably 0.1 MPa or higher, more preferably 0.13 MPa or higher. In the light of economical efficiency and safety, a reaction pressure is preferably 2 MPa or lower, more preferably 0.99 MPa or lower.

A gas space velocity is preferably 2000 hr⁻¹ or more, more preferably 4000 hr⁻¹ or more, in the light of economical efficiency. A gas space velocity is preferably 60000 hr⁻¹ or less, more preferably 30000 hr⁻¹ or less, in the light of activity and selectivity.

A gas fed into a reactor can be dividedly fed to the reactor so as to control a reaction temperature.

The reaction can be conducted in a fixed bed, a fluidized bed, a moving bed or the like, and can be preferably selected, taking both of control of a reaction temperature and a regeneration method of the catalyst into account. For example, a fixed bed may include a quench type reactor such as an internal multistage quench type, a multitubular type reactor, a multistage type reactor having a plurality of internal heat exchangers or the like, a multistage cooling radial flow type, a double pipe heat exchange type, an internal cooling coil type, a mixed flow type, and other types of reactors.

When used, a catalyst for synthesizing an olefin-containing gas can be diluted with silica, alumina or an inert and stable heat conductor for controlling a temperature. In addition, when used, a catalyst for synthesizing an olefin-containing gas can be applied to the surface of a heat exchanger for controlling a temperature.

In the step of separation in the 4th process for producing LPG, water, unreacted materials, i.e., methanol and/or dimethyl ether, or the like are separated from the olefin-containing gas obtained in the above step of producing an olefin-containing gas by a known method, as necessary, and then an ethylene-containing fraction is separated from it to obtain a propylene-containing fraction.

The propylene-containing fraction comprises an olefin mixture containing propylene or butene, more preferably propylene, as a main component.

On the other hand, the ethylene-containing fraction, which is separated, comprises an olefin mixture containing ethylene as a main component. It may comprise other components, but a higher content of C2 components (ethylene and ethane) is preferable; specifically, it is preferably 80% by weight or more.

Furthermore, a butene-containing fraction, which comprises an olefin mixture containing butene as a main component, can be separated before or after separating the ethylene-containing fraction from the olefin-containing gas, for obtaining a propylene-containing fraction with a higher content of propylene. The olefin-containing gas may be separated into a propylene-containing fraction, an ethylene-containing fraction and a butene-containing fraction in one operation. By separating a butene-containing fraction, a content of propylene in a propylene-containing fraction may increase, and consequently, a content of propane in LPG produced may increase.

In this invention, a substance having a lower boiling point than the boiling point of propylene (a low-boiling-point component) is an ethylene-containing fraction, while a substance having a higher boiling point than the boiling point of propane (a high-boiling-point component) is a butene-containing fraction.

An ethylene-containing fraction can be separated by a known method, for example, gas-liquid separation, absorption separation or distillation; more specifically, gas-liquid separation at an ambient temperature under increased pressure, absorption separation at an ambient temperature under increased pressure, gas-liquid separation with cooling, absorption separation with cooling, or combination thereof Alternatively, for this purpose, membrane separation or adsorption separation can be conducted, or these in combination with gas-liquid separation, absorption separation or distillation can be conducted. A gas recovery process commonly employed in an oil factory (described in “Oil Refining Processes”, ed. The Japan Petroleum Institute, Kodansha Scientific, 1998, pp. 28-32) can be applied to separation of an ethylene-containing fraction.

The separation conditions may be determined as appropriate in accordance with a known method.

Furthermore, a low-boiling-point component other than ethylene can be separated from the resulting ethylene-containing fraction by a known method, for the purpose of recycling an ethylene-containing fraction with a higher content of ethylene as a starting material for the step of producing an olefin-containing gas

When separating a butene-containing fraction, the butene-containing fraction can be reacted with hydrogen to produce a liquefied petroleum gas containing butane as a main component, after a high-boiling-point component other than butene and butane is separated from the butene-containing fraction by a known method, as necessary. And, like an ethylene-containing fraction, a part or all of the butene-containing fraction can be recycled as a starting material for the step of producing an olefin-containing gas.

It is preferable that the total content of propylene, propane, butene and butane is higher in a propylene-containing fraction fed to the step of hydrogenating an olefin-containing gas; specifically, it is preferably 70% by weight or more. Particularly, it is preferable that the total content of propylene and propane is higher in a propylene-containing fraction fed to the step of hydrogenating an olefin-containing gas; specifically, it is preferably 50% by weight or more.

In the step of recycling in the 4th process for producing LPG, a part or all of the ethylene-containing fraction, which is separated from the olefin-containing gas in the above step of separation, is recycled as a starting material for the step of producing an olefin-containing gas.

In the light of economical efficiency, the whole ethylene-containing fraction separated in the step of separation is preferably recycled to the step of producing an olefin-containing gas. Alternatively, part of the ethylene-containing fraction may be removed outside the system, while the rest of ethylene-containing fraction may be recycled to the step of producing an olefin-containing gas.

For the purpose of recycling an ethylene-containing fraction separated in the step of separation, a known technique, e.g., appropriately providing a recycle line with a pressurization means can be employed.

In the step of hydrogenating an olefin-containing gas in the 4th process for producing LPG, a liquefied petroleum gas containing propane or butane as a main component is produced from hydrogen and the propylene-containing fraction obtained in the above step of separation, in the presence of a catalyst for hydrogenating an olefin-containing gas.

In the step of hydrogenating an olefin-containing gas, propane is produced by reacting propylene with hydrogen, following the formula (III) shown below; while butane is produced by reacting butene with hydrogen, following the formula (IV) shown below.
C₃H₆+H₂→C₃H₈ (III)
C₄H₈+H₂→C₄H₁₀ (IV)

Examples of a catalyst for hydrogenating an olefin-containing gas include the ones described above. In the 4th process for producing LPG, similarly, a preferable catalyst for hydrogenating an olefin-containing gas is a nickel catalyst, a palladium catalyst, a platinum catalyst and the like.

In the 4th process for producing LPG, similarly, a catalyst for hydrogenating an olefin-containing gas may be used singly or in combination of two or more types. A catalyst for hydrogenating an olefin-containing gas may comprise other additive components as long as its intended effect would not be impaired. For example, the above catalyst may be diluted with quartz sand and the like, and used.

There may be two or more catalyst layers comprising a catalyst for hydrogenating an olefin-containing gas. In the catalyst layer comprising a catalyst for hydrogenating an olefin-containing gas, the composition may change in regard to the direction of flowing of the raw material gas.

The reaction can be conducted in a fixed bed, a fluidized bed or a moving bed. When there are two or more catalyst layers, it is preferable to conduct the reaction in a fixed bed. The reaction conditions such as a composition of a raw material gas, a reaction temperature, a reaction pressure and a contact time with a catalyst may be appropriately determined, in accordance with a known method, depending on the type, the performance and the shape of the used catalyst, and the like.

When using Pd-alumina (Palladium supported alumina) as a catalyst for hydrogenating an olefin-containing gas, for example, the reaction may be carried out under the following conditions.

A content of a propylene-containing fraction in a gas fed into a reactor may be appropriately determined, depending on the composition (content of propylene, content of butene) of the propylene-containing fraction and the like, and it may be, for example, 10 to 80% by weight.

A ratio of hydrogen to a propylene-containing fraction in a gas fed into a reactor may be appropriately determined, depending on the composition (content of propylene, content of butene) of the propylene-containing fraction and the like. Generally, a ratio (by mole) of hydrogen to olefin (mainly, propylene and butene) in a gas fed into a reactor is preferably 1.1 (H₂/CH₂ₙ) or more, more preferably 1.5 (H₂/CₙH₂ₙ) or more, in the light of hydrogenating an olefin more sufficiently. A ratio (by mole) of hydrogen to olefin (mainly, propylene and butene) in a gas fed into a reactor is preferably 10 (H₂/CₙH₂ₙ) or less, more preferably 5 (H₂/CₙH₂ₙ) or less, in the light of economical efficiency.

A gas fed into a reactor may comprise water, an inert gas and the like, in addition to a propylene-containing fraction and hydrogen.

The propylene-containing fraction obtained in the step of separation, and hydrogen may be mixed, and then fed into the reactor or, alternatively, these may be fed into the reactor separately. And, a gas fed into a reactor can be dividedly fed to the reactor.

In the light of activity, a reaction temperature is preferably 120° C. or higher, more preferably 140° C. or higher. In the light of selectivity and removing reaction heat, a reaction temperature is preferably 400° C. or lower, more preferably 350° C. or lower

In the light of activity, a reaction pressure is preferably 0.11 MPa or higher, more preferably 0.13 MPa or higher. In the light of economical efficiency and safety, a reaction pressure is preferably 3 MPa or lower, more preferably 2 MPa or lower.

A gas space velocity is preferably 1000 hr⁻¹ or more, more preferably 1500 hr⁻¹ or more, in the light of economical efficiency. A gas space velocity is preferably 40000 hr⁻¹ or less, more preferably 20000 hr⁻¹ or less, in the light of activity.

Next, there will be described an embodiment of a process for producing LPG according to this invention (the 4th process for producing LPG) with reference to the drawings.

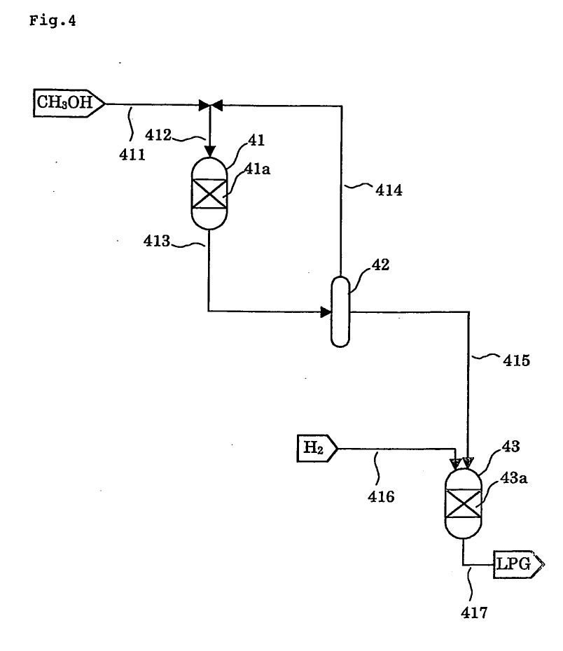

FIG. 4 shows an embodiment of an LPG production apparatus suitable for carrying out the 4th process for producing LPG according to this invention.

First, methanol and/or dimethyl ether, which are reaction raw materials, is fed into a first reactor 41 via lines 411 and 412. In addition, an ethylene-containing fraction recycled as a starting material is fed into the first reactor 41 from a separator 42 via a recycle line 414 and the line 412. In the first reactor 41, there is a catalyst for synthesizing an olefin-containing gas 41a. In the first reactor 41, in the presence of the catalyst for synthesizing an olefin-containing gas 41a, a reaction gas comprising an olefin mixture containing propylene or butene as a main component and ethylene as well as water is synthesized from methanol and/or dimethyl ether and the ethylene-containing fraction.

The obtained reaction gas, which comprises an olefin mixture containing propylene or butene as a main component and ethylene as well as water, is fed into a separator 42 via a line 413, after removing water or the like by, for example, gas-liquid separation. In the separator 42, the reaction gas after removing water or the like, i.e., the synthesized olefin-containing gas is separated into a propylene-containing fraction comprising propylene or butene as a main component, and an ethylene-containing fraction comprising ethylene as a main component, which has a lower boiling point than the boiling point of propylene (a low-boiling-point component).

A butene/butane-containing fraction comprising butene or butane as a main component, which has a higher boiling point than the boiling point of propane (a high-boiling-point component) can be separated from the synthesized olefin-containing gas.

The separated ethylene-containing fraction is recycled into the first reactor 41 via the recycle line 414 and the line 412.

The propylene-containing fraction separated in the separator 42 is fed into a second reactor 43 via a line 415. And, hydrogen is fed into the second reactor 43 via a line 416. In the second reactor 43, there is a catalyst for hydrogenating an olefin-containing gas 43a. In the second reactor 43, in the presence of the catalyst for hydrogenating an olefin-containing gas 43a, the olefin mixture containing propylene or butene as a main component is hydrogenated to produce a paraffin mixture containing propane or butane as a main component.

The synthesized paraffins are pressurized and cooled, and LPG, which is a product, is obtained from a line 417. Hydrogen, methane, ethane, olefins having five carbon atoms or more, naphtha, oils and the like may be removed from the LPG by a known method, for example, gas-liquid separation.

The LPG production apparatus may be, as necessary, provided with a booster, a heat exchanger, a valve, an instrumentation controller and so on, which are not shown.

Thus, LPG is produced from at least one selected from the group consisting of methanol and dimethyl ether, in the 4th process for LPG production according to this invention.

According to the process for LPG production of this invention, LPG containing propane or butane as a main component; specifically, LPG with the total content of propane and butane of 90% or more, specifically 95% or more (including 100%) on the basis of carbon can be produced.

And, according to the process for LPG production of this invention, LPG containing propane as a main component; specifically, LPG with a content of propane of 50% or more, specifically 60% or more, more specifically 90% or more (including 100%) on the basis of carbon can be produced. LPG produced according to the present invention has a composition suitable for a propane gas, which is widely used as a fuel for household and business use.

Methanol, which is used as a raw material in this invention, is produced in an industrial and large scale from a synthesis gas produced by, for example, a water-vapor reforming method, a complex reforming method and an autothermal reforming method of a natural gas (methane), or from a water gas produced from a coal coke. The present invention, wherein LPG is produced from methanol, is expected as an industrially practicable process for producing LPG.

### EXAMPLES

The following will describe the present invention in more detail with reference to Examples. However, the present invention is not limited to these Examples.

### Example 1

An LPG was prepared using the LPG production apparatus shown in FIG. 1. The used catalyst for synthesizing an olefin-containing gas was a catalyst prepared by mixing 73.5% by dry weight of H-ZSM-5 with a Si/Al ratio (atomic ratio) of 25.0 and 26.5% by dry weight of alumina binder (Cataloid AP; produced by Catalysts & Chemicals Ind. Co., Ltd.), and then wet molding, drying and calcining the mixture. The used catalyst for hydrogenating an olefin-containing gas was a 2.0 wt % Pt/carbon catalyst (produced by N.E. ChemCat Corporation). Both the catalyst for synthesizing an olefin-containing gas and the catalyst for hydrogenating an olefin-containing gas were a 1/32-inch cylindrical extrusion-molded article having the same shape.

A raw material gas with a composition of 50 mol % of methanol and 50 mol % of hydrogen was passed through a catalyst layer comprising a first catalyst layer consisting of the above catalyst for synthesizing an olefin-containing gas as the front half (50 volume % of the reactor), and a second catalyst layer consisting of a granular mixture of the above catalyst for hydrogenating an olefin-containing gas and the above catalyst for synthesizing an olefin-containing gas, with a volume ratio of 1:1, as the rear half (50 volume % of the reactor). The reaction conditions were as follows; as reaction temperature, the inlet control temperature of a reactor was 330° C. and the maximum temperature of a catalyst layer was 375° C.; the reaction pressure: partial pressure of methanol was 70 kPa; the liquid space velocity of methanol to the catalyst for synthesizing an olefin-containing gas was 40 hr⁻¹; the liquid space velocity of methanol to the catalyst for hydrogenating an olefin-containing gas was 40 hr⁻¹; therefore, the liquid space velocity of methanol to all the catalysts was 20 hr⁻¹.

From gas chromatographic analysis of the product, unreacted methanol was not detected, and the conversion reaction of methanol substantially completely proceeded. A conversion of methanol to an LPG, i.e., to propane and butane was 66% on the basis of carbon, and a content of propane in the LPG was 64% on the basis of carbon.

### Example 2

An LPG was prepared using the LPG production apparatus shown in FIG. 2. The used Catalysts for synthesizing an olefin-containing gas were the catalyst for synthesizing an olefin-containing gas used in Example 1, and a catalyst in which 0.2 wt % Pt was supported on the catalyst for synthesizing an olefin-containing gas used in Example 1 (hereinafter, referred to as a “catalyst for isomerizing and hydrogenating an olefin-containing gas”). The used catalyst for hydrogenating an olefin-containing gas was the catalyst for hydrogenating an olefin-containing gas used in Example 1. This catalyst for isomerizing and hydrogenating an olefin-containing gas is a catalyst for synthesizing an olefin-containing gas having catalytic function for hydrogenating an olefin.

A raw material gas with the same composition as in Example 1 was passed through a first catalyst layer consisting of the above catalyst for synthesizing an olefin-containing gas in ⅔ (by volume) part from the inlet, and the above catalyst for isomerizing and hydrogenating an olefin-containing gas in the remaining ⅓ part. The reaction conditions were as follows; as reaction temperature, the inlet control temperature of a reactor was 330° C. and the maximum temperature of a catalyst layer was 375° C.; the reaction pressure: partial pressure of methanol was 70 kPa; the liquid space velocity of methanol to the catalyst in the first catalyst layer was 25 hr⁻¹.

Next, the resulting reaction gas was passed through a second catalyst layer consisting of the above catalyst for hydrogenating an olefin-containing gas. The reaction conditions were as follows; the reaction temperature was 330° C.; the reaction pressure was 110 kPa; the liquid space velocity to the catalyst for hydrogenating an olefin-containing gas based on the raw material methanol was 100 hr⁻¹.

From gas chromatographic analysis of the product, unreacted methanol was not detected, and the conversion reaction of methanol substantially completely proceeded. A conversion of methanol to an LPG, i.e., to propane and butane was 68% on the basis of carbon, and a content of propane in the LPG was 70% on the basis of carbon.

### Example 3

An LPG was prepared using the LPG production apparatus shown in FIG. 3. The used catalyst for synthesizing an olefin-containing gas was the catalyst for synthesizing an olefin-containing gas used in Example 1. The used Catalysts for hydrogenating an olefin-containing gas were the catalyst for hydrogenating an olefin-containing gas used in Example 1, and the catalyst for isomerizing and hydrogenating an olefin-containing gas used in Example 2.

A raw material gas with a composition of 50 mol % of methanol and 50 mol % of steam was passed through a first catalyst layer consisting of the above catalyst for synthesizing an olefin-containing gas. The reaction conditions were as follows; as reaction temperature, the inlet control temperature of a reactor was 330° C. and the maximum temperature of a catalyst layer was 365° C.; the reaction pressure: partial pressure of methanol was 70 kPa; the liquid space velocity of methanol to the catalyst in the first catalyst layer was 40 hr⁻¹.

Next, the resulting reaction gas and hydrogen were passed through a second catalyst layer consisting of the above catalyst for isomerizing and hydrogenating an olefin-containing gas in ½ (by volume) part from the inlet, and the above catalyst for hydrogenating an olefin-containing gas in the remaining ½ part. An amount of hydrogen supplied to the reaction gas was equal (by mole) to the amount of the raw material methanol gas fed into the first catalyst layer. The reaction conditions were as follows; the reaction temperature was 330° C.; the reaction pressure was 120 kPa; the liquid space velocity to the catalyst for hydrogenating an olefin-containing gas based on the raw material methanol was 40 hr⁻¹.

From gas chromatographic analysis of the product, unreacted methanol was not detected, and the conversion reaction of methanol substantially completely proceeded. A conversion of methanol to an LPG, i.e., to propane and butane was 70% on the basis of carbon, and a content of propane in the LPG was 67% on the basis of carbon.

### Reference Example 1

In a tubular reactor was charged a cylindrical extrusion-molded catalyst with a diameter of 0.8 mm consisting of 65 wt % of a proton-type ZSM-5 with a SiO₂/Al₂O₃ ratio of 50 (Si/Al ratio (atomic ratio) is 25) and 35 wt % of alumina binder, and then a raw material gas with a composition of methanol:hydrogen=1:1 (molar ratio) was passed through the reactor to carry out the reaction. The reaction conditions were as follows; the inlet control temperature of a reactor was 330° C. (603 K); the reaction pressure was 0.14 MPa; the gas space velocity of methanol was 11200 hr⁻¹. The product was analyzed by gas chromatography. The results are shown in Table 1.

### Reference Example 2

The reaction was carried out in the same way as Reference Example 1, except that a raw material gas with a composition of ethylene hydrogen=1:2 (molar ratio) was used, and the gas space velocity of ethylene was equal to the gas space velocity of methanol in Reference Example 1 on the basis of carbon (the gas space velocity of ethylene:5600 hr⁻¹). The results are shown in Table 1.

### Reference Example 3

The reaction was carried out in the same way as Reference Example 1, except that a raw material gas with a composition of methanol:ethylene:hydrogen=2:1:4 (molar ratio) was used, and the total of the gas space velocity of methanol and the gas space velocity of ethylene was equal to the gas space velocity of methanol in Reference Example 1 on the basis of carbon (the gas space velocity of methanol:5600 hr⁻¹ and the gas space velocity of ethylene:2800 hr⁻¹). The results are shown in Table 1.

### Reference Example 4

The reaction was carried out in the same way as Reference Example 1, except that a raw material gas with a composition of methanol:hydrogen=1:1 (molar ratio) was used, and the gas space velocity of methanol was 5600 hr⁻¹. Separately, the reaction was carried out in the same way as Reference Example 2, except that a raw material gas with a composition of ethylene:hydrogen=1:2 (molar ratio) was used, and the gas space velocity of ethylene was 2800 hr⁻¹. The resulting products were mixed by a ratio of 2:1. In other words, the reaction was carried out in the same way as Reference Example 3, except that methanol and ethylene were separately passed through a tubular reactor. The results are shown in Table 1.

TABLE 1

| | Reference | Reference | Reference | Reference |
|---|---|---|---|---|
| | Example 1 | Example 2 | Example 3 | Example 4 |
| conversion (%) | 90.0 | 13.6 | 53.6 | 48.2 |
| Product composition (%) | | | | |
| C1 (methane) | 0.8 | 1.4 | 0.7 | 1.0 |
| C2 (ethane, ethylene) | 17.4 | 5.7 | 0.6 | 1.0 |
| C3 (propane, propylene) | 34.2 | 39.4 | 50.7 | 41.2 |
| C4 (butane, butene) | 46.8 | 53.5 | 47.6 | 56.0 |
| C5 (pentane, pentene) | 0.8 | 0.0 | 0.4 | 0.8 |
| C3 + C4 | 81.0 | 92.9 | 98.3 | 97.2 |
| C3/C3 + C4 | 42.2 | 42.4 | 51.6 | 42.4 |

The results of Reference Examples 1 to 4 shown in Table 1 indicate that a reaction gas comprising C3 components (propylene and propane) or C4 components (butene and butane) as main components can be obtained from methanol, from ethylene or from a mixture of methanol and ethylene. Particularly, Reference Example 3, where a mixture of methanol and ethylene was used as a raw material, gave a reaction gas comprising C3 components (propylene and propane) as main components.

### Example 4

An LPG was prepared using the LPG production apparatus shown in FIG. 4.

The used catalyst for synthesizing an olefin-containing gas was a cylindrical extrusion-molded catalyst with a diameter of 0.8 mm consisting of 65 wt % of a proton-type ZSM-5 with a SiO₂/Al₂O₃ ratio of 50 (Si/Al ratio (atomic ratio) is 25) and 35 wt % of alumina binder. The used catalyst for hydrogenating an olefin-containing gas was a cylindrical 2.0 wt % Pt/carbon catalyst with a diameter of 0.8 mm (produced by N.E. ChemCat Corporation).

(Olefin-Containing Gas Production Step)

A raw material gas with a composition of methanol: an ethylene-containing fraction, which was separated from the olefin-containing gas and recycled as a starting material for the step of producing an olefin-containing gas (described later)=2:1 (molar ratio) was passed through a layer of the above catalyst for synthesizing an olefin-containing gas. The reaction conditions were as follows; the inlet control temperature of a reactor was 330° C.; the reaction pressure was 0.14 MPa; the gas space velocity of methanol was 5600 hr⁻¹.

Gas chromatographic analysis of the product (olefin-containing gas) indicated that it had a composition of 31 wt % of a low-boiling-point component, 57 wt % of an LPG fraction and 12 wt % of a heavy fraction, and that a conversion of methanol was 100%.

(Separation/Recycle Step)

The olefin-containing gas obtained in the olefin-containing gas production step was subjected to gas-liquid separation, and then dried over molecular sieves. The olefin-containing gas was subjected to separation with cooling, to separate a gas consisting of 2 wt % of methane, 1 wt % of ethane and 97 wt % of ethylene as an ethylene-containing fraction (low-boiling-point component), giving a propylene-containing fraction.

Gas chromatographic analysis of the propylene containing fraction indicated that it had a composition of 7 wt % of propane, 56 wt % of propylene, 11 wt % of butane, 21 wt % of butene and 5 wt % of other components.

The separated ethylene-containing fraction was recycled as a starting material for the olefin-containing gas production step.

(Olefin-Containing Gas Hydrogenation Step)

Subsequently, the propylene-containing fraction obtained in the separation/recycle step and hydrogen were passed through a layer of the above catalyst for hydrogenating an olefin-containing gas. A supplied amount of hydrogen was 4.5 times as much as the propylene-containing fraction (by mole) [substantially equal (by mole) to the amount of the raw material methanol gas passed through the layer of the catalyst for synthesizing an olefin-containing gas]. The reaction conditions were as follows; the reaction temperature was 250° C.; the reaction pressure was 0.14 MPa; the gas space velocity of the propylene-containing fraction was 5600 hr⁻¹.

Gas chromatographic analysis of the product indicated that it had a composition of 63 wt % of propane, 32 wt % of butane and 5 wt % of other components. On the basis of carbon, 78% of the methanol fed was converted into an LPG in which the total content of propane and butane was 95% and a content of propane was 63%.

### Comparative Example 1

An LPG was prepared in the same way as Example 4, except that the ethylene-containing fraction separated in the separation step was not recycled as a starting material for the olefin-containing gas production step; the gas space velocity of methanol was 5600 hr⁻¹ in the olefin-containing gas production step; and the supplied amount of hydrogen in the olefin-containing gas hydrogenation step was equal (by mole) to that of the raw material methanol gas passed through the layer of the catalyst for synthesizing an olefin-containing gas.

Gas chromatographic analysis of the product in the olefin-containing gas production step (olefin-containing gas) indicated that it had a composition of 23 wt % of a low-boiling-point component, 57 wt % of an LPG fraction and 20 wt % of a heavy fraction, and that a conversion of methanol was 96%.

Gas chromatographic analysis of the product in the olefin-containing gas hydrogenation step indicated that it had a composition of 1 wt % of methane, 34 wt % of ethane, 36 wt % of propane, 19 wt % of butane and 10 wt % of other components. On the basis of carbon, only 68% of the methanol fed was converted into an LPG and the total content of propane and butane in the LPG was 81%.

### INDUSTRIAL APPLICABILITY

As described above, according to this invention, a liquefied petroleum gas containing propane or butane as a main component can be produced from at least one selected from the group consisting of methanol and dimethyl ether.

In addition, according to this invention, a liquefied petroleum gas containing propane or butane as a main component can be more economically produced from at least one selected from the group consisting of methanol and dimethyl ether.

1. A process for producing a liquefied petroleum gas, comprising a step of:
producing a liquefied petroleum gas containing propane or butane as a main component from hydrogen and at least one selected from the group consisting of methanol and dimethyl ether by a catalytic reaction.

2. A process for producing a liquefied petroleum gas, comprising a step of:
producing a liquefied petroleum gas containing propane or butane as a main component by passing a raw material gas comprising hydrogen and at least one selected from the group consisting of methanol and dimethyl ether through a catalyst layer.

3. The process for producing a liquefied petroleum gas according to claim 2, wherein the catalyst layer comprising, in the direction of flowing of the raw material gas,
a former catalyst layer comprising a catalyst for synthesizing an olefin-containing gas which is used when producing an olefin-containing gas from at least one selected from the group consisting of methanol and dimethyl ether, and
a latter catalyst layer comprising a catalyst for hydrogenating an olefin-containing gas which is used when an olefin is hydrogenated to produce a paraffin.

4. The process for producing a liquefied petroleum gas according to claim 2, wherein the catalyst layer comprising, in the direction of flowing of the raw material gas,
a former catalyst layer comprising a catalyst for synthesizing an olefin-containing gas which is used when producing an olefin-containing gas from at least one selected from the group consisting of methanol and dimethyl ether,
a middle catalyst layer comprising a zeolite catalyst component and a catalyst component for hydrogenating an olefin which is used when an olefin is hydrogenated to produce a paraffin, and
a latter catalyst layer comprising a catalyst for hydrogenating an olefin-containing gas which is used when an olefin is hydrogenated to produce a paraffin.

5. A process for producing a liquefied petroleum gas, comprising:
(1) a step of producing an olefin-containing gas wherein a raw material gas comprising hydrogen and at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component, water and hydrogen; and
(2) a step of hydrogenating an olefin-containing gas wherein the reaction gas obtained in the step of producing an olefin-containing gas is passed through a catalyst layer comprising a catalyst for hydrogenating an olefin-containing gas which is used when an olefin is hydrogenated to produce a paraffin, to produce a liquefied petroleum gas containing propane or butane as a main component.

6. A process for producing a liquefied petroleum gas, comprising:
(1) a step of producing an olefin-containing gas wherein a raw material gas comprising hydrogen and at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component, water and hydrogen; and
(2) a step of isomerizing and hydrogenating an olefin-containing gas wherein the reaction gas obtained in the step of producing an olefin-containing gas is passed through a catalyst layer comprising a zeolite catalyst component and a catalyst component for hydrogenating an olefin which is used when an olefin is hydrogenated to produce a paraffin, to produce a liquefied petroleum gas containing propane or butane as a main component.

7. A process for producing a liquefied petroleum gas, comprising:
(1) a step of producing an olefin-containing gas wherein a raw material gas comprising hydrogen and at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component, water and hydrogen;
(2) a step of isomerizing and hydrogenating an olefin-containing gas wherein the reaction gas obtained in the step of producing an olefin-containing gas is passed through a catalyst layer comprising a zeolite catalyst component and a catalyst component for hydrogenating an olefin-containing gas which is used when an olefin is hydrogenated to produce a paraffin, to produce a reaction gas containing propylene or butene as a main component and propane or butane and hydrogen; and
(3) a step of hydrogenating an olefin-containing gas wherein the reaction gas obtained in the step of isomerizing and hydrogenating an olefin-containing gas is passed through a catalyst layer comprising a catalyst for hydrogenating an olefin-containing gas which is used when an olefin is hydrogenated to produce a paraffin, to produce a liquefied petroleum gas containing propane or butane as a main component.

8. A process for producing a liquefied petroleum gas, comprising:
(1) a step of producing an olefin-containing gas wherein a raw material gas comprising at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component and water; and
(2) a step of hydrogenating an olefin-containing gas wherein the reaction gas obtained in the step of producing an olefin-containing gas and a hydrogen-containing gas are passed through a catalyst layer comprising a catalyst for hydrogenating an olefin-containing gas which is used when an olefin is hydrogenated to produce a paraffin, to produce a liquefied petroleum gas containing propane or butane as a main component.

9. A process for producing a liquefied petroleum gas, comprising:
(1) a step of producing an olefin-containing gas wherein a raw material gas comprising at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component and water; and
(2) a step of isomerizing and hydrogenating an olefin-containing gas wherein the reaction gas obtained in the step of producing an olefin-containing gas and a hydrogen-containing gas are passed through a catalyst layer comprising a zeolite catalyst component and a catalyst component for hydrogenating an olefin-containing gas which is used when an olefin is hydrogenated to produce a paraffin, to produce a liquefied petroleum gas containing propane or butane as a main component.

10. A process for producing a liquefied petroleum gas, comprising:
(1) a step of producing an olefin-containing gas wherein a raw material gas comprising at least one selected from the group consisting of methanol and dimethyl ether is passed through a catalyst layer comprising a catalyst for synthesizing an olefin-containing gas which is used when producing an olefin-containing gas from at least one selected from the group consisting of methanol and dimethyl ether, to obtain a reaction gas comprising an olefin mixture containing propylene or butene as a main component and water;
(2) a step of isomerizing and hydrogenating an olefin-containing gas wherein the reaction gas obtained in the step of producing an olefin-containing gas and a hydrogen-containing gas are passed through a catalyst layer comprising a zeolite catalyst component and a catalyst component for hydrogenating an olefin which is used when an olefin is hydrogenated to produce a paraffin, to produce a reaction gas containing propylene or butene as a main component and propane or butane and hydrogen; and
(3) a step of hydrogenating an olefin-containing gas wherein the reaction gas obtained in the step of isomerizing and hydrogenating an olefin-containing gas is passed through a catalyst layer comprising a catalyst for hydrogenating an olefin-containing gas which is used when an olefin is hydrogenated to produce a paraffin, to produce a liquefied petroleum gas containing propane or butane as a main component.

11. A process for producing a liquefied petroleum gas, comprising:
(1) a step of producing an olefin-containing gas wherein an olefin-containing gas comprising propylene or butene as a main hydrocarbon component as well as ethylene is produced from at least one selected from the group consisting of methanol and dimethyl ether and the ethylene-containing fraction which is separated from the olefin-containing gas in the step of separation and recycled as a starting material for the step of producing an olefin-containing gas in the step of recycling, in the presence of a catalyst for synthesizing an olefin-containing gas which is used when producing an olefin-containing gas from at least one selected from the group consisting of methanol and dimethyl ether;
(2) a step of separation wherein an ethylene-containing fraction is separated from the olefin-containing gas obtained in the step of producing an olefin-containing gas, to obtain a propylene-containing fraction;
(3) a step of recycling wherein a part or all of the ethylene-containing fraction separated in the step of separation is recycled as a starting material for the step of producing an olefin-containing gas; and
(4) a step of hydrogenating an olefin-containing gas wherein a liquefied petroleum gas containing propane or butane as a main component is produced from hydrogen and the propylene-containing fraction obtained in the step of separation, in the presence of a catalyst for hydrogenating an olefin-containing gas which is used when an olefin is hydrogenated to produce a paraffin.

12. The process for producing a liquefied petroleum gas according to claim 1, wherein the content of propane in the liquefied petroleum gas produced is 50 to 100% on the basis of carbon.

13. The process for producing a liquefied petroleum gas according to claim 1, wherein the total content of propane and butane in the liquefied petroleum gas produced is 90 to 100% on the basis of carbon.
